(19) United States
(12) Patent Application Publication
(10) Pub. No.: US 2007/0191647 A1
(43) Pub. Date: Aug. 16, 2007

(54) Polyalkylene glycol lubricant base oils with narrow molar mass distribution

(75) Inventors: Klaus Poellmann, Burghausen (DE); Anton Strasser, Altoetting (DE); Guido Scholz, Burgkirchen (DE); Reinhard Vybiral, Burgkirchen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 11/705,944

(22) Filed: Feb. 13, 2007

(30) Foreign Application Priority Data
Feb. 14, 2006 (DE) 102006006696.0

(51) Int. Cl. C07C 31/18

(52) U.S. Cl. 568/866

(57) ABSTRACT

The present invention provides a process for preparing polyalkylene glycols by reacting a mixture of a mono- or polyhydric alcohol and a polyalkylene glycol prepolymer with a C₂- to C₄-alkylene oxide or a mixture of such alkylene oxides in the presence of a catalyst which comprises Zn₃[Co(CN)₆]₂, the mono- or polyhydric alcohol having a molecular weight of up to 500 g/mol and containing from 1 to 20 carbon atoms and from 1 to 10 hydroxyl groups, and the polyalkylene glycol prepolymer being the addition product of a C₂- to C₄-alkylene oxide or of a mixture of such alkylene oxides in an amount of at least 500 g/mol to a mono- or polyhydric alcohol having from 1 to 20 carbon atoms and from 1 to 10 hydroxyl groups.

The present invention relates to a process for preparing polyalkylene glycols with narrow molar mass distribution and low content of low molecular weight, allyl-functional by-products starting from propylene oxide or mixtures of ethylene oxide and propylene oxide, which are usable as lubricant base oils.

Polyalkylene glycols are prepared generally by anionic, ring-opening polymerization of alkylene oxides (ethylene oxide, propylene oxide, butylene oxide) with alcohols as initiators according to the reaction equation below (see Ullmann Encyclopedia of Industrial Chemistry 5. ed VCH, ISBN 3-527-20100-9). When R′—OH=butanol, this forms α-butoxy-Ω-hydroxypolyalkylene glycols. When R′—OH=diethylene glycol or dipropylene glycol as an initiator, this forms α,Ω-dihydroxypolyalkylene glycols. The use and requirements of these polyalkylene glycols as base oils for formulating lubricants is, for example, described in the FDA Regulations 21 CFR 178.3910 (surface lubricants used in manufacture of metallic articles) or 21 CFR 173.340, 21 CFR 178.3570 (Lubricants with incidental food contact). These polyalkylene glycol base oils are characterized by means of their viscosity, their mean molar mass and molar mass distribution, and the ratio of the incorporated alkylene oxide monomers. The catalysts used for the anionic polymerization of the alkylene oxides are generally alkali metal hydroxides such as potassium hydroxide or sodium hydroxide.

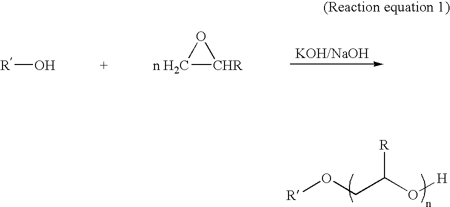

It is known that, with propylene oxide as the monomer, the alkali metal hydroxide catalysts mentioned not only catalyze the chain growth of the polymers formed but simultaneously promote the rearrangement of the propylene oxide to allyl alcohol (reaction equation 2) (de Luca, Rodriguez, Perez-Collado in Polym. Int. 51, 1066-1071).

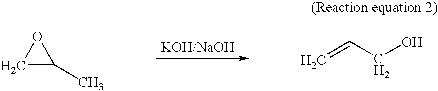

Since the allyl alcohol formed acts as an initiator R″—OH of the anionic polymerization according to reaction equation 1 and competes with the original initiator alcohol and the growing polyalkylene glycol chain, the molar mass distribution widens, and the mean molar mass and the viscosity of the resulting polyalkylene glycol base oils decrease. Simultaneously formed are low molecular weight allyl-functional polyalkylene glycols which increase the proportion of volatile organic substances (VOCs) and hence the evaporation loss in high-temperature applications. These allyl-functional polyalkylene glycol base oils can be determined in a simple manner by means of ¹H NMR or by means of iodine number titration by standard method DGF C-V 11b.

Attempts have been made for some time to suppress this rearrangement reaction as far as possible. A slight but insufficient improvement is possible by the use of cesium hydroxide instead of potassium hydroxide or sodium hydroxide and the reduction in the reaction temperature, which, though, slows the polymerization overall and hence makes it less economically viable (JP-A-10 110 029).

The mean molar mass can be determined analytically by determining the OH number to DIN 53240. This gives only the number average (Mn). The more significant molar mass distribution is obtained, for example, by means of gel permeation chromatography (GPC) with polyethylene glycol standards and THF as the solvent on a combination of 10², 10³, 10⁴ Å DVB gel columns. The width of the molar mass distribution is reported by the so-called polydispersity D=Mw/Mn, in which Mw=weight-average molar mass and Mn=number-average molar mass. The closer Mw/Mn is to 1.0, the narrower is the molar mass distribution and generally the smaller are also the low molecular weight fractions. The proportion of low molecular weight, volatile substances (VOCs=volatile organic compounds) can additionally also be determined by means of gas chromatography, for example by means of the method according to DIN 55649 (determination of the content of volatile organic compounds) or in a simpler manner by determining the evaporation loss at different temperatures.

Also in the case of preparation of polyether polyols as raw materials of polyurethane foam synthesis, the formation of allyl alcohol as a rearrangement product of propylene oxide and consequently the formation of monools in the alkaline catalysis with alkali metal hydroxides is a great problem. For this reason, polyether polyols for polyurethane foam synthesis have increasingly been prepared in recent times using, as well as alkali metal hydroxides, so-called DMC catalysts of the formula Zn₃[Co(CN)₆]₂.xZnCl₂.yH₂O.z glyme, which are said to significantly reduce the formation of allyl-functional monools and significantly increase the reaction rate. The preparation and use of these catalysts is, for example, described in EP-A-555 053 (Example 1), EP-A-700 949, EP-A-743 093, EP-A-968 055, EP-A-1 244 519, EP-A-1 276 563.

For the use of polyalkylene glycols as lubricant base oils, the viscosity is a crucial criterion. Therefore, base oils for lubricants are divided into so-called ISO-VG classes (viscosity class according to DIN 51519).

From the polyalkylene glycols as base oils, it is possible by addition of suitable additives to formulate finished lubricants, for example transmission oils or hydraulic oils, which then have increased demands with regard to wear behavior, lubrication performance and aging stability. A typical test of the characterization of the lubrication performance is the FZG test to DIN 51354.

It was an object of the present invention to provide polyalkylene glycol base oils for the formulation of lubricants, which, in comparison to the conventional types which have been prepared by means of anionic polymerization, have a lower content of allyl groups, a narrower molar mass distribution and smaller proportions of low molecular weight, volatile components (VOCs), and hence exhibit lower losses in use as a lubricant at the predefined use temperatures.

It has now been found that, surprisingly, polyalkylene glycols suitable for use as base oils for lubricants can be prepared by alkoxylating a polyalkylene glycol prepolymer with a single alkylene oxide or a mixture of a plurality of alkylene oxides by means of zinc-cobalt dimetal cyanide catalysis (DMC catalysis) in the presence of a mono- or polyhydric alcohol.

The present invention thus provides a process for preparing polyalkylene glycols by reacting a mixture of a mono- or polyhydric alcohol and a polyalkylene glycol prepolymer with a C₂- to C₄-alkylene oxide or a mixture of such alkylene oxides in the presence of a catalyst which comprises Zn₃[Co(CN)₆]₂, the mono- or polyhydric alcohol having a molecular weight of up to 500 g/mol and containing from 1 to 20 carbon atoms and from 1 to 10 hydroxyl groups, and the polyalkylene glycol prepolymer being the addition product of a C₂- to C₄-alkylene oxide or of a mixture of such alkylene oxides in an amount of at least 500 g/mol to a mono- or polyhydric alcohol having from 1 to 20 carbon atoms and from 1 to 10 hydroxyl groups.

The present invention further provides polyalkylene glycols obtainable by the above-described process.

The invention further provides for the use of polyalkylene glycols obtainable by the above-described process as a base oil for producing lubricants.

The mono- or polyhydric alcohol having from 1 to 20 carbon atoms and from 1 to 10 hydroxyl groups (which is referred to hereinafter as “starter alcohol”) has a molecular weight of up to 500 g/mol, i.e. is monomeric and distillable, and contains, in a preferred embodiment, from 2 to 12 carbon atoms. It preferably also contains from 1 to 6, in particular 1, 2, 3 or 4, hydroxyl groups. It may also comprise one, two, three or four (C₂-C₄)-alkoxy groups.

Preferred starter alcohols are, for example

monoalcohols of the formula R′—OH where R′═C₁-C₂₀-alkyl, R′═C₁-C₂₀-alkyl-O—(C₂-C₄)-alkylene- (for example methylglycol, butylglycol), R′═C₁-C₂₀-alkyl-O—(C₂-C₄)-alkylene-O—(C₂-C₄)-alkylene (for example methyldiglycol, butyldiglycol), R′═C₁-C₂₀-alkyl-O—(C₂-C₄)-alkylene-O—(C₂-C₄)-alkylene-O—(C₂-C₄)-alkylene (for example methyltriglycol, butyltriglycol),

diols of the formula HO—R²—OH where R²═C₂-C₆-alkylene, (C₂-C₄)-alkylene-O—(C₂-C₄)-alkylene or (C₂-C₄)-alkylene-O—(C₂-C₄)-alkylene-O—(C₂-C₄)-alkylene (for example diethylene glycol, dipropylene glycol, triethylene glycol, tripropylene glycol, tetraethylene glycol, tetrapropylene glycol),

triols, for example glycerol and trimethylolpropane, and

tetraols, for example pentaerythritol.

Particular preference is given to monoalcohols R′—OH where R′═C₁-C₄-alkyl, and the corresponding C₁-C₄-alkyl ethers of monoethylene glycol, monopropylene glycol, diethylene glycol, dipropylene glycol, triethylene glycol or tripropylene glycol.

The polyalkylene glycol prepolymers are α-alkoxy-Ω-hydroxy copolymers and α,Ω-dihydroxy copolymers, which have been prepared from ethylene oxide, propylene oxide and/or butylene oxide or mixtures thereof in any composition and a mono- or polyhydric alcohol having from 1 to 20 carbon atoms and from 1 to 10 hydroxyl groups or water. Preference is given to those polyalkylene glycol prepolymers which contain propylene oxide in a proportion of at least 20% by weight.

The alkylene oxide groups present in the polyalkylene glycol prepolymer together have at least a molecular weight of 500 g/mol. In a preferred embodiment, the polyalkylene glycol prepolymer comprises at least one alkylene oxide chain having a molecular weight of 500 g/mol.

In a preferred embodiment, the polyalkylene glycol prepolymer is an addition product of C₂- to C₄-alkylene oxide or a mixture of such alkylene oxides to an alcohol having from 2 to 12 carbon atoms. The alcohol also preferably contains from 1 to 6, in particular 1, 2, 3 or 4, hydroxyl groups.

In a further preferred embodiment, the polyalkylene glycol prepolymer corresponds to the formula (1)

R³O-(A-O)ₐ—H]_{b} (1),

in which

a is from 5 to 70,

b is from 1 to 10,

(A-O) is a C₂- to C₄-alkoxy group,

R³ is a hydrocarbon group having from 1 to 20 carbon atoms and from 1 to 10 hydroxyl groups.

In a further preferred embodiment, the polyalkylene glycol prepolymer corresponds to the formula (2)

R⁵—O-(A-O)ₐ—R⁴ (2)

in which

a is from 5 to 70,

(A-O) is a C₂- to C₄-alkoxy group, and

R⁴ and R⁵ are each independently hydrogen or a C₁- to C₆-alkyl group, but at least one of the R⁴ or R⁵ radicals is hydrogen.

The zinc-cobalt dimetal cyanide catalyst used (also referred to hereinafter as “DMC catalyst”) is known from the prior art. It comprises Zn₃[Co(CN)₆]₂.

The process according to the invention is preferably performed as follows.

The starter alcohol is preferably dissolved in a polyalkylene glycol prepolymer in a pressure reactor in a concentration of from 1 to 40% by weight, in particular from 5 to 30% by weight. The pulverulent DMC catalyst is dispersed homogeneously in this mixture in a concentration of preferably 100-600 ppm. The reaction mixture is heated under nitrogen to a temperature of preferably from 90 to 170° C., in particular to a temperature of 110-150° C. At this temperature, a portion of the alkylene oxide is metered in. After onset of the polymerization reaction, recognizable by the pressure drop and the temperature increase in the reactor, the remaining amount of the alkylene oxide necessary to achieve the particular viscosity of the lubricant base oil is metered in.

The inventive polyalkylene glycols thus obtained are accordingly α-alkoxy-Ω-hydroxy copolymers, α,Ω-dihydroxy copolymers, α,Ω-trihydroxy copolymers and α,Ω-tetrahydroxy copolymers of the monomers ethylene oxide, propylene oxide and/or butylene oxide in any composition. Preferred lubricant base oils are those polyalkylene glycols which contain propylene oxide in a proportion of at least 20% by weight. Also preferred are those polyalkylene glycols which have a mean molar mass of 700-50 000 g/mol, preferably 700-10 000 g/mol. Preference is also given to those polyalkylene glycols which, at 40° C., have kinematic viscosities of from 20 to 40 000 mm²/s, preferably from 20 to 6000 mm²/s.

The inventive polyalkylene glycols generally contain from 10 to 100 ppm of DMC catalyst and have a narrow molar mass distribution with small low molecular weight volatile fractions.

A further advantage of the process according to the invention for preparing polyalkylene glycols is the significantly shorter reaction time which is required to achieve a certain viscosity in comparison to the preparation process with alkaline catalysis.

Depending on the ratio of the alkylene oxides used, the inventive polyalkylene glycols are more or less water-soluble. By adding customary lubricant additives and corrosion inhibitors, for example phosphoric esters, or sulfur compounds such as mercaptobenzothiazole or triazoles and aminic corrosion inhibitors, it is possible to formulate, from the inventive polyalkylene glycol lubricant base oils, ready-formulated lubricants for various uses, for example transmission oils, hydraulic oils, lubricating greases or metalworking fluids.

In addition, it is also possible to add customary antioxidants in amounts of up to 5000 ppm to the polyalkylene glycols to prepare ready-formulated lubricants. Typical antioxidants are, for example, phenolic antioxidants such as BHT, para-methoxyphenol, hydroquinone or BHA, or aminic antioxidants such as polymeric trimethyldihydroquinoline.

### EXAMPLES

### Example 1

### Inventive

Preparation of an α-butoxy-Ω-hydroxypolypropylene glycol having a molar mass of 2500 g/mol, polyalkylene glycol lubricant base oil of viscosity class ISO VG 150

A 5 l pressure reactor is initially charged with 754 g of a mixture of an α-butoxy-Ω-hydroxypolypropylene glycol (80% by weight) with molar mass 800 g/mol and butoxydipropylene glycol (20% by weight). Under nitrogen, 160 ppm of the DMC catalyst described in EP 1 276 563 are added to this mixture. The mixture is heated to a temperature of 130° C. under nitrogen, and an amount of 3468 g of propylene oxide is metered in at a pressure of about 2 bar at such a rate that the heat of reaction which arises can be removed. The period of metered addition is about 2 hours. The α-butoxy-Ω-hydroxypolypropylene glycol formed exhibits the following characteristics: molar mass (number-average from GPC) Mn=2500 g/mol, molar mass (weight-average from GPC) Mw=2600 g/mol, polydispersity=Mw/Mn=1.04, OH number=21.8 mgKOH/g, kinematic viscosity at 40° C.=181 mm²/s, kinematic viscosity at 100° C.=31 mm²/s, density at 20° C.=0.996 g/cm³. ¹H NMR end group analysis: ratio of butyl:sec-OH:allyl=1:1:0.01. The content of VOCs (volatile organic compounds) determined to DIN 55649 is 0.98 g/liter.

### Example 2

### Inventive

Preparation of an α-butoxy-Ω-hydroxypolypropylene glycol having a molar mass of 3400 g/mol, polyalkylene glycol lubricant base oil of viscosity class ISO VG 320

A 5 l pressure reactor is initially charged with 343 g of a mixture of an α-butoxy-Ω-hydroxypolypropylene glycol (80% by weight) with molar mass 800 g/mol and butoxydipropylene glycol (20% by weight). Under nitrogen, 200 ppm of the DMC catalyst described in EP 1 276 563 are added to this mixture. The mixture is heated to a temperature of 130° C. under nitrogen, and an amount of 2780 g of propylene oxide is metered in at a pressure of about 2 bar at such a rate that the heat of reaction which arises can be removed. The period of metered addition is about 3 hours. The α-butoxy-Ω-hydroxypolypropylene glycol formed exhibits the following characteristics: OH number=15.6 mgKOH/g, molar mass (number-average from GPC) Mn=3400 g/mol, molar mass (weight-average from GPC) Mw=4000 g/mol, polydispersity=1.18, kinematic viscosity at 40° C.=360 mm²/s, kinematic viscosity at 100° C.=58 mm²/s, density at 20° C.=0.998 g/cm³. ¹H NMR end group analysis: ratio of butyl:sec-OH:allyl=1:1.05:0.01. The content of VOCs (volatile organic compounds) determined to DIN 55649 is 0.98 g/liter.

### Example 3

### Inventive

Preparation of an α,Ω-dihydroxypolyoxyethylene-oxypropylene glycol copolymer having a molar mass of 2000 g/mol, polyalkylene glycol lubricant base oil of viscosity class ISO VG 220

A 5 l pressure reactor is initially charged with 551 g of a mixture of an α,Ω-dihydroxypolyoxyethylene-oxypropylene glycol copolymer (90% by weight) with molar mass 2000 g/mol and dipropylene glycol (10% by weight). Under nitrogen, 400 ppm of the DMC catalyst described in EP 1 276 563 are added to this mixture. The mixture is heated to a temperature of 145° C. under nitrogen, and an amount of 783 g of a mixture of ethylene oxide and propylene oxide (ratio 64:36% by weight) is metered in within a period of 3 hours at a pressure of about 2 bar at such a rate that the heat of reaction which arises can be removed. The α,Ω-dihydroxypolyoxyethylene-oxypropylene glycol copolymer formed exhibits the following characteristics: OH number=53.4 mgKOH/g, molar mass (number-average from GPC) Mn=2000 g/mol, molar mass (weight-average from GPC) Mw=2350 g/mol, polydispersity=1.18, kinematic viscosity at 40° C.=225 mm²/s, kinematic viscosity at 100° C.=43 mm²/s, density at 20° C.=1.085 g/cm³. ¹H NMR end group analysis: ratio of prim-OH:sec-OH:allyl=0.34:1:0.01. The content of VOCs (volatile organic compounds) determined to DIN 55649 is 0.96 g/liter.

### Example 4

### Use of the Product from Example 1 in a Lubricant Formulation

At from 60 to 90° C., 0.5% of isotridecyl phosphate, 0.5% of phenolic antioxidant (4,4-methylenebis(2,6-di-tert-butylphenol)), 300 ppm of tolyltriazole and 3.5% of N-phenylstyrenized benzylamine are dissolved in the product from Example 1. A transmission lubricant of ISO VG (viscosity class to DIN 51519) 150 is obtained, which achieves load state 12 in the FZG test to DIN 51354.

### Comparative Example 1

Preparation of an α-butoxy-Ω-hydroxypolypropylene glycol having a molar mass of 3300 g/mol by means of alkaline catalysis; conventionally prepared polyalkylene glycol lubricant base oil of viscosity class ISO VG 320

A 5 l pressure reactor is initially charged with 20.8 g of butoxydipropylene glycol and 4.4 g of potassium hydroxide. Application of reduced pressure at approx. 100° C. for 1 hour dries the mixture. Subsequently, the mixture is heated to a temperature of 130° C. under nitrogen, and an amount of 1966 g of propylene oxide is metered in at a pressure of from about 3 to 6 bar at such a rate that the heat of reaction which arises can be removed. The period of metering is about 30 hours. The α-butoxy-Ω-hydroxypolypropylene glycol formed exhibits the following characteristics: molar mass (number-average from GPC) Mn=3300 g/mol, molar mass (weight-average from GPC) Mw=5400 g/mol, polydispersity=Mw/Mn=1.31, OH number=20 mgKOH/g, kinematic viscosity at 40° C.=372 mm²/s, kinematic viscosity at 100° C.=60 mm²/s, density at 20° C.=1.00 g/cm³. ¹H NMR end group analysis: ratio of butyl:sec-OH:(allyl+propenyl)=1:2.08:0.7. The content of VOCs (volatile organic compounds) determined to DIN 55649 is 1.95 g/liter.

The formation of low molecular weight fractions here significantly broadens the molar mass distribution and increases the polydispersity. Likewise, more low molecular weight volatile fractions (VOCs) are formed. For this reason, the reaction time which is required to achieve the given ISO VG class is also significantly longer.

1. A process for preparing polyalkylene glycols by reacting in an alkoxylation reaction a mixture of a mono- or polyhydric alcohol and a polyalkylene glycol prepolymer with a C₂- to C₄-alkylene oxide or a mixture of such alkylene oxides in the presence of a DMC catalyst which comprises Zn₃[Co(CN)₆]₂, the mono- or polyhydric alcohol having a molecular weight of up to 500 g/mol and containing from 1 to 20 carbon atoms and from 1 to 10 hydroxyl groups, and the polyalkylene glycol prepolymer being the addition product of a C₂- to C₄-alkylene oxide or of a mixture of such alkylene oxides in an amount of at least 500 g/mol to a mono- or polyhydric alcohol having from 1 to 20 carbon atoms and from 1 to 10 hydroxyl groups.

2. The process as claimed in claim 1, in which a weight ratio of the mixture of polyalkylene glycol prepolymer and mono- or polyhydric alcohol before the alkoxylation reaction is between 99:1 and 60:40.

3. The process as claimed in claim 1, wherein the DMC catalyst is present in a concentration of 100-600 ppm.

4. The process of claim 1, in which the polyalkylene glycol prepolymer is an addition product of C₂- to C₄-alkylene oxide or a mixture of such alkylene oxides to an alcohol having from 2 to 12 carbon atoms and from 1 to 6 hydroxyl groups.

5. The process of claim 1, in which the polyalkylene glycol prepolymer of the formula 2
R⁵—O-(A-O)ₐ—R⁴ (2)
in which
a is from 5 to 70,
(A-O) is a C₂- to C₄-alkoxy group, and
R⁴ and R⁵ are each independently hydrogen or a C₁- to C₆-alkyl group, but at least one of the R⁴ or R⁵ radicals is hydrogen and
contains propylene oxide in a proportion of at least 20% by weight as a monomer.

6. The process of claim 1, in which the starter alcohol contains from 2 to 12 carbon atoms and from 1 to 6 hydroxyl groups.

7. The process of claim 1, in which the starter alcohol R′—OH is selected from monoalcohols of the formula R′—OH where R′═C₁-C₂₀-alkyl or R′═C₁-C₂₀-alkyl-(O—(C₂-C₄)-alkylene)ₙ- where n=1, 2, or 3, diols of the formula HO—R²—OH where R²═C₂-C₆-alkylene, —(C₂-C₄)-alkylene-O—(C₂-C₄)-alkylene- or —(C₂-C₄)-alkylene-O—(C₂-C₄)-alkylene-O—(C₂-C₄)-alkylene-, glycerol, trimethylolpropane or pentaerythritol.

8. The process of claim 1, in which the C₂-C₄-alkylene oxide or mixture thereof is selected from the group consisting of ethylene oxide, propylene oxide, butylene oxide, and mixtures thereof.

9. The process of claim 1, in which the alkoxylation reaction is effected at a temperature of 90-170° C.

10. A polyalkylene glycol prepared by a process comprising:
reacting in an alkoxylation reaction a mixture of a mono- or polyhydric alcohol and a polyalkylene glycol prepolymer with a C₂— to C₄-alkylene oxide or a mixture of such alkylene oxides in the presence of a DMC catalyst which comprises Zn₃[Co(CN)₆]₂, the mono- or polyhydric alcohol having a molecular weight of up to 500 g/mol and containing from 1 to 20 carbon atoms and from 1 to 10 hydroxyl groups, and the polyalkylene glycol prepolymer being the addition product of a C₂— to C₄-alkylene oxide or of a mixture of such alkylene oxides in an amount of at least 500 g/mol to a mono- or polyhydric alcohol having from 1 to 20 carbon atoms and from 1 to 10 hydroxyl groups.

11. The polyalkylene glycol as claimed in claim 10 which has a mean molar mass of from 700 to 50 000 g/mol and a kinematic viscosity at 40° C. of between 20 and 40 000 mm²/s.

12. The polyalkylene glycol as claimed in claim 10 which has a polydispersity D=Mw/Mn by GPC analysis of less than 1.25.

13. The polyalkylene glycol of claim 10, in which the volatile fractions determined to DIN 55649 are less than 1200 ppm.

14. A lubricant base oil comprising the polyalkylene glycol of claim 10.

15. An aqueous hydraulic fluid comprising the polyalkylene glycol of claim 10.

16. A lubricant base oil consisting of the polyalkylene glycol of claim 10, customary lubricant additives, antioxidants, and corrosion inhibitors.
